SEQUENCE LISTING (5 sequences)

SEQ ID NO: 1 - DNA, 19 bp, Artificial Sequence
  siRNA against nociceptin/orphanin FQ peptide (NOP) receptor
tcattgctatcgactacta

SEQ ID NO: 2 - DNA, 19 bp, Artificial Sequence
  siRNA against nociceptin/orphanin FQ peptide (NOP) receptor
agacagctaccaacattta

SEQ ID NO: 3 - DNA, 19 bp, Artificial Sequence
  siRNA against rat Gbeta2
caactgaagaaccaaatta

SEQ ID NO: 4 - DNA, 19 bp, Artificial Sequence
  siRNA against rat Gbeta2
acatctgggatgccatgaa

SEQ ID NO: 5 - DNA, 19 bp, Artificial Sequence
  siRNA against rat Gbeta4
acaacatctgctccatata

(19) United States
(12) Patent Application Publication
(10) Pub. No.: US 2011/0229972 A1
(43) Pub. Date: Sep. 22, 2011

(54) COMPOSITIONS AND METHODS FOR MATERIAL TRANSFER INTO CELLS

(75) Inventor: Victor Ruiz-Velasco, Hershey, PA (US)

(73) Assignee: The Penn State Research Foundation, University Park, PA (US)

(21) Appl. No.: 13/052,621

(22) Filed: Mar. 21, 2011

Related U.S. Application Data
(60) Provisional application No. 61/315,538, filed on Mar. 19, 2010.

(51) Int. Cl. C12N 15/87; C12N 13/00

(52) U.S. Cl. 435/461; 435/173.6

(57) ABSTRACT

Methods for material transfer into a cell are provided according to the present invention which include electroporation of the cell in the presence of the material, such as nucleic acids, and 2,3-butanedione monoxime; and lipofection with a complex of a lipid-based carrier, such as liposomes, and the material, in the presence of 2,3-butanedione monoxime.

### REFERENCE TO RELATED APPLICATION

This application claims priority from U.S. Provisional Patent Application Ser. No. 61/315,538, filed Mar. 19, 2010, the entire content of which is incorporated herein by reference.

### GOVERNMENT SPONSORSHIP

This invention was made with government support under Grant No. DA025574 awarded by the National Institutes of Health and National Institute on Drug Abuse. The government has certain rights in the invention.

### FIELD OF THE INVENTION

The present invention relates generally to compositions and methods for transfer of a material into cells. In specific embodiments, the present invention relates to compositions and methods for nucleic acid transfer into cells.

### SUMMARY OF THE INVENTION

Methods for material transfer into a cell are provided according to the present invention which include electroporation of the cell in the presence of the material and 2,3-butanedione monoxime (BDM); and contacting the cell with a complex of a lipid-based carrier and the material, in the presence of 2,3-butanedione monoxime.

Methods for nucleic acid transfer into a cell are provided according to the present invention which include electroporation of the cell in the presence of the nucleic acid and 2,3-butanedione monoxime; and contacting the cell with a complex of a lipid-based carrier and the nucleic acid, in the presence of 2,3-butanedione monoxime.

Methods for nucleic acid transfer into a cell are provided according to the present invention which include electroporation of the cell in the presence of the nucleic acid and 2,3-butanedione monoxime; and contacting the cell with a complex of a liposome and the nucleic acid, in the presence of 2,3-butanedione monoxime.

Methods for material transfer into a cell are provided according to the present invention which include electroporation of the cell in the presence of the material and 2,3-butanedione monoxime; and contacting the cell with a complex of a lipid-based carrier and the material, in the presence of 2,3-butanedione monoxime, wherein the steps of 1) electroporation and 2) contacting the cell with a complex of a lipid-based carrier and the material, are performed in any order or simultaneously. Each of the electroporation and contacting steps can be performed more than one time. According to embodiments of the present invention, each of the steps of 1) electroporation and 2) contacting the cell with a complex of a lipid-based carrier and the material, is performed at least two times on the same cells.

According to methods described herein, a nucleic acid transferred into a cell can be DNA or RNA. According to methods described herein, a nucleic acid transferred into a cell can be inhibitory RNA. According to methods described herein, a nucleic acid transferred into a cell can be siRNA and/or shRNA.

According to methods described herein, a material is transferred into a eukaryotic cell by electroporation of the eukaryotic cell in the presence of the material and 2,3-butanedione monoxime; and contacting the eukaryotic cell with a complex of a lipid-based carrier and the material.

According to methods described herein, a material is transferred into an excitable cell by electroporation of the excitable cell in the presence of the material and 2,3-butanedione monoxime; and contacting the excitable cell with a complex of a lipid-based carrier and the material.

According to methods described herein, a material is transferred into a neuron by electroporation of the neuron in the presence of the material and 2,3-butanedione monoxime; and contacting the neuron with a complex of a lipid-based carrier and the material.

Methods for material transfer into a cell in vitro are provided according to the present invention which include electroporation of the cell in the presence of the material and 2,3-butanedione monoxime; and contacting the cell with a complex of a lipid-based carrier and the material, in the presence of 2,3-butanedione monoxime.

Cells containing a material transferred into the cell according to a method of the present invention are provided as described herein.

Cells containing a nucleic acid transferred into the cell according to a method of the present invention are provided as described herein.

Methods for material transfer into a cell in vitro are provided according to the present invention which include electroporation of the cell in vitro in the presence of the material and 2,3-butanedione monoxime; contacting the cell in vitro with a complex of a lipid-based carrier and the material, in the presence of 2,3-butanedione monoxime; and transfer of the cell containing the material into an animal.

Methods for nucleic acid transfer into a cell are provided according to the present invention which include electroporation of the cell in vitro in the presence of the nucleic acid and 2,3-butanedione monoxime; contacting the cell in vitro with a complex of a liposome and the nucleic acid, in the presence of 2,3-butanedione monoxime; and transfer of the cell containing the nucleic acid into an animal.

Kits for material transfer into a cell are provided according to the present invention which include an electroporation reagent; 2,3-butanedione monoxime; and a lipid-based carrier.

Kits for material transfer into a cell are provided according to the present invention which include an electroporation buffer which is a cell-compatible buffer such as, but not limited to, DMEM; 2,3-butanedione monoxime; and a lipid-based carrier.

### BRIEF DESCRIPTION OF THE DRAWINGS

FIG. 1A is an image taken by phase contrast microscopy showing stellate ganglion (SG) primary neurons after electroporation with 500 nM Cy3-tagged siRNA in the presence of 2 mM BDM;

FIG. 1B is an image taken by fluorescence microscopy showing the same field of SG neurons shown in FIG. 1A after electroporation with 500 nM Cy3-tagged siRNA in the presence of 2 mM BDM;

FIG. 1C is an image taken by phase contrast microscopy showing SG neurons after electroporation with 500 nM Cy3-tagged siRNA in the presence of 2 mM BDM;

FIG. 1D is an image taken by fluorescence microscopy showing the same field of SG neurons shown in FIG. 1C after electroporation with 500 nM Cy3-tagged siRNA in the presence of 2 mM BDM;

FIG. 2A is an image taken by phase contrast microscopy showing SG neurons after electroporation with 500 nM Cy3-tagged siRNA in the presence of 2 mM BDM;

FIG. 2B is an image taken by fluorescence microscopy showing the same field of SG neurons shown in FIG. 2A;

FIG. 2C is an image taken by phase contrast microscopy showing SG neurons after electroporation with 500 nM Cy3-tagged siRNA in the presence of 2 mM BDM;

FIG. 2D is an image taken by fluorescence microscopy showing the same field of SG neurons shown in FIG. 2C;

FIG. 2E is an image taken by phase contrast microscopy showing SG neurons after electroporation with 500 nM Cy3-tagged siRNA in the presence of 2 mM BDM;

FIG. 2F is an image taken by fluorescence microscopy showing the same field of SG neurons shown in FIG. 2E;

FIG. 2G is an image taken by phase contrast microscopy showing SG neurons after electroporation in the presence of 2 mM BDM and four hour lipofection with 500 nM Cy3-tagged siRNA in the presence of 2 mM BDM;

FIG. 2H is an image taken by fluorescence microscopy showing the same field of SG neurons shown in FIG. 2G;

FIG. 2I is an image taken by phase contrast microscopy showing SG neurons after electroporation in the presence of 2 mM BDM and four hour lipofection with 500 nM Cy3-tagged siRNA in the presence of 2 mM BDM;

FIG. 2J is an image taken by fluorescence microscopy showing the same field of SG neurons shown in FIG. 2I;

FIG. 2K is an image taken by phase contrast microscopy showing SG neurons after electroporation in the presence of 2 mM BDM and four hour lipofection with 500 nM Cy3-tagged siRNA in the presence of 2 mM BDM;

FIG. 2L is an image taken by fluorescence microscopy showing the same field of SG neurons shown in FIG. 2K;

FIG. 3A is an image taken by phase contrast microscopy showing SG neurons after electroporation and one hour lipofection with 500 nM Cy3-tagged siRNA in the presence of 2 mM BDM;

FIG. 3B is an image taken by fluorescence microscopy showing the same field of SG neurons shown in FIG. 3A;

FIG. 3C is an image taken by phase contrast microscopy showing SG neurons after electroporation and one hour lipofection with 500 nM Cy3-tagged siRNA in the presence of 2 mM BDM;

FIG. 3D is an image taken by fluorescence microscopy showing the same field of SG neurons shown in FIG. 3C;

FIG. 3E is an image taken by phase contrast microscopy showing SG neurons after electroporation and one hour lipofection with 500 nM Cy3-tagged siRNA in the presence of 2 mM BDM;

FIG. 3F is an image taken by fluorescence microscopy showing the same field of SG neurons shown in FIG. 3E;

FIG. 3G is an image taken by phase contrast microscopy showing SG neurons after electroporation and two hour lipofection with 500 nM Cy3-tagged siRNA in the presence of 2 mM BDM;

FIG. 3H is an image taken by fluorescence microscopy showing the same field of SG neurons shown in FIG. 3G;

FIG. 3I is an image taken by phase contrast microscopy showing SG neurons after electroporation and two hour lipofection with 500 nM Cy3-tagged siRNA in the presence of 2 mM BDM;

FIG. 3J is an image taken by fluorescence microscopy showing the same field of SG neurons shown in FIG. 3I;

FIG. 3K is an image taken by phase contrast microscopy showing SG neurons after electroporation and two hour lipofection with 500 nM Cy3-tagged siRNA in the presence of 2 mM BDM;

FIG. 3L is an image taken by fluorescence microscopy showing the same field of SG neurons shown in FIG. 3K;

FIG. 4A is an image taken by phase contrast microscopy showing SG neurons after electroporation without siRNA in the presence of 2 mM BDM followed by four hour lipofection with 500 nM Cy3-tagged siRNA in the presence of 2 mM BDM;

FIG. 4B is an image taken by fluorescence microscopy showing the same field of SG neurons shown in FIG. 4A;

FIG. 4C is an image taken by phase contrast microscopy showing SG neurons after electroporation without siRNA in the presence of 2 mM BDM followed by four hour lipofection with 500 nM Cy3-tagged siRNA in the presence of 2 mM BDM;

FIG. 4D is an image taken by fluorescence microscopy showing the same field of SG neurons shown in FIG. 4C;

FIG. 4E is an image taken by phase contrast microscopy showing SG neurons after electroporation without siRNA in the presence of 2 mM BDM followed by four hour lipofection with 500 nM Cy3-tagged siRNA in the presence of 2 mM BDM;

FIG. 4F is an image taken by fluorescence microscopy showing the same field of SG neurons shown in FIG. 4E;

FIG. 5A is an image of a Western blot showing the effects of transfection of SG neurons in vitro with siRNA oligonucleotides directed against the nociceptin/orphanin FQ peptide (NOP) receptor by electroporation+BDM alone, lipofection+BDM alone, or a combination of electroporation+BDM and lipofection+BDM, compared to a control;

FIG. 5B is a graph showing the effects of transfection of SG neurons in vitro with siRNA oligonucleotides directed against NOP receptors by a combination of electroporation+BDM and lipofection+BDM, compared to a control;

FIG. 6 is a graph showing the effects of transfection of SG neurons in vitro with siRNA oligonucleotides directed against NOP by electroporation+BDM alone, lipofection+BDM alone, or a combination of electroporation+BDM and lipofection+BDM, compared to a control;

FIG. 7A is a graph showing the effects of transfection of SG neurons in vitro with siRNA oligonucleotides directed against the G protein Gβ2 by a combination of electroporation+BDM and lipofection+BDM, compared to a control;

FIG. 7B is an image showing the effects of transfection of SG neurons in vitro with siRNA oligonucleotides directed against the G protein Gβ2 by a combination of electroporation+BDM and lipofection+BDM, compared to a control;

FIG. 7C is a graph showing the effects of transfection of SG neurons in vitro with siRNA oligonucleotides directed against the G protein Gβ2 by a combination of electroporation+BDM and lipofection+BDM, compared to a control;

FIG. 8A is a graph showing the effects of transfection of SG neurons in vitro with siRNA oligonucleotides directed against the G protein Gβ2 and the G protein Gβ4 by a combination of electroporation+BDM and lipofection+BDM, compared to a control;

FIG. 8B is an image showing the effects of transfection of SG neurons in vitro with siRNA oligonucleotides directed against the G protein Gβ2 and the G protein Gβ4 by a combination of electroporation+BDM and lipofection+BDM, compared to a control; and

FIG. 8C is a graph showing the effects of transfection of SG neurons in vitro with siRNA oligonucleotides directed against the G protein Gβ2 and the G protein Gβ4 by a combination of electroporation+BDM and lipofection+BDM, compared to a control.

### DETAILED DESCRIPTION OF THE INVENTION

The present invention relates generally to compositions and methods for transfer of a material into cells. In specific embodiments, the present invention relates to compositions and methods for nucleic acid transfer into cells. Methods, compositions and kits of the present invention are useful in various applications, including, but not limited to, production of gene products in cells and research applications.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, Pa., 2003; and Herdewijn, P. (Ed.), Oligonucleotide Synthesis: Methods and Applications, Methods in Molecular Biology, Humana Press, 2004.

The singular terms “a,” “an,” and “the” are not intended to be limiting and include plural referents unless explicitly state or the context clearly indicates otherwise.

Methods for transfer of a material into a cell are provided according to embodiments of the present invention that include electroporation of the cell in the presence of the material desired to be delivered into the cell and 2,3-butanedione monoxime; and contacting the cell with a complex of: a lipid-based carrier and the material desired to be delivered into the cell; and 2,3-butanedione monoxime.

Methods for nucleic acid transfer into a cell are provided according to embodiments of the present invention that include electroporation of the cell in the presence of the nucleic acid and 2,3-butanedione monoxime; and lipofection of the cell with a complex of a lipid-based carrier and the nucleic acid; and 2,3-butanedione monoxime.

The reagent 2,3-butanedione monoxime is typically included in a concentration in the range of 0.01 millimolar-100 millimolar. In farther embodiments, 2,3-butanedione monoxime is typically included in a concentration in the range of 0.1 millimolar-50 millimolar. In still further embodiments, 2,3-butanedione monoxime is typically included in a concentration in the range of 0.25 millimolar-25 millimolar.

A synergistic effect on delivery of materials into cells using combined electroporation and lipofection methods is found when performed in the presence of 2,3-butanedione monoxime according to embodiments of methods of the present invention. In particular, the percentage of cells into which the material is delivered is increased using methods of the present invention compared to methods using electroporation or lipofection alone.

A synergistic effect on delivery of nucleic acids into cells using combined electroporation and lipofection methods is found when performed in the presence of 2,3-butanedione monoxime according to embodiments of methods of the present invention. In particular, the transfection efficiency is increased compared to methods using electroporation or lipofection alone. The term ‘transfection efficiency’ refers to the percentage of cells transfected.

Methods according to embodiments of the present invention can be used to transfer material into a variety of cell types, including dividing and non-dividing cell types. In particular embodiments, methods according to embodiments of the present invention can be used to transfer material into excitable cells, such as myocytes and neurons. Cells in primary culture or cell lines can be used. Cells can be dissociated cells or non-dissociated cells such as cell aggregates or tissues.

Methods according to embodiments of the present invention can be used to transfer material into cells in vitro, such as but not limited to, primary culture or cell lines.

Once the material is transferred into a cell using methods of the present invention, the cell can be transferred to an animal. Methods according to embodiments of the present invention include, electroporation of a cell in the presence of the material and 2,3-butanedione monoxime; contacting the cell with a complex of a lipid-based carrier and the material, in the presence of 2,3-butanedione monoxime; and transfer of the cell to an animal.

For example, using well-known methodology, one or more stem cells is introduced into a non-human host embryo and the embryo with the introduced stem cells is then gestated under suitable conditions, such as by introduction into a pseudopregnant female animal. The term “stem cell” refers to pluripotent stem cells, such as embryonic stem (ES) cells, and induced pluripotent stem (iPS) cells.

Methods according to embodiments of the present invention can be used to transfer material into cells from any of a variety of species, including but not limited to, mammals, birds, reptiles, amphibians, fish, bacteria and yeast. Methods according to embodiments of the present invention can be used to transfer material into mammalian cells including, but not limited to, human, non-human primate, rodent, horse, cow, pig, sheep, goat and rabbit cells. Methods according to embodiments of the present invention can be used to transfer material into rodent cells including, but not limited to, mouse, rat and guinea pig cells.

The term “electroporation” refers to application of an electric voltage to cells in the presence of a material desired to be delivered inside cells, temporarily allowing cell membranes to become porous to passage of materials, such as nucleic acids, into the cells. Conditions used for electroporation include selection of voltage used, pulse width and number of pulses. Typically, a voltage in the range of about 800 V/cm-1400 V/cm is applied in a pulse of about 8 milliseconds-15 milliseconds. More than one pulse can be applied, typically 1-3 pulses are applied. Particular conditions selected depend on variables such as cell type, size and species from which the cell is derived and such conditions are selected by one of skill in the art. Electroporation methods are well-known in the art, for example, as described in J. A. Nickoloff, Animal Cell Electroporation and Electrofusion Protocols, Humana Press; 1st ed., 1995.

Preferred electroporation methods used in embodiments of the present invention use a “capillary” electroporation system such as described in Kim J A, et al., 2008, Biosens Bioelectron 23(9):1353-1360; and US Patent Publication 2007/0275454. A commercially available “capillary” electroporation system is the NEON Transfection System, from Invitrogen, Inc, USA.

The term “lipofection” refers to methods of introducing a nucleic acid into cells by contacting the cells with complexes including a lipid-based carrier and the nucleic acid. Cationic liposomes are an example of a lipid-based carrier that complexes with nucleic acids.

The term “lipid-based carrier” refers to macromolecular structures having lipid and/or lipid derivatives as the major constituent.

The term “material” is used herein to refer to a substance to be delivered into a cell. Such materials can be any of a variety of useful biologically active molecules and substances including, but not limited to, proteins, peptides, carbohydrates, oligosaccharides, drugs, and nucleic acids capable of being complexed with a lipid-based carrier. The term “biologically active molecules and substances” refers molecules or substances that exert a biological effect in vitro and/or in vivo, such as, but not limited to, nucleic acids, inhibitory RNA, siRNA, shRNA, ribozymes, antisense nucleic acids, antibodies, hormones, small molecules, aptamers, decoy molecules, toxins and chemotherapeutics.

Lipids included in lipid-based carriers can be naturally-occurring lipids, synthetic lipids or combinations thereof. Cationic lipids are an example of lipids included alone or in combination with other lipids or additives as a lipid-based carrier for polyanionic materials, such as, but not limited to, nucleic acids. The term cationic lipid refers to any lipid which has a net positive charge at physiological pH.

Well-known cationic lipids included in compositions, kits and methods of the invention include, but are not limited to N-(1-(2,3-dioleyloxy)propyl)-N,N,N-trimethylammonium chloride (DOTMA); 1,2-dioleoyloxy-3-(trimethylammonium)propane (DOTAP); 1,2-dioleoyl-3-dimethylammonium-propane (DODAP); dioctadecylamidoglycylspermine (DOGS), commercially as Transfectam® (Promega, Madison, Wis.); 1,2-dipalmitoylphosphatidylethanolamidospermine (DPPES); 2,3-dioleyloxy-N-(2-(sperminecarboxamido)ethyl)-N,N-dimethyl-1-propanaminium trifluoroacetate (DOSPA); dimyristoyltrimethylammonium propane (DMTAP); (3-dimyristyloxypropyl)(dimethyl)(hydroxyethyl)ammonium (DMRIE); dioctadecyldimethylammonium chloride (DODAC), Dimethyldidodecylammonium bromide (DDRB); 3β[N—(N′,N′-dimethylaminoethane)-carbamoyl]cholesterol (DC-Chol); 1-[2-(9(Z)-octadecenoyloxy)-ethyl]-2-(8(Z)-heptadecenyl)-3-(2-hydroxyethyl)-imidazolinium (DOTIM); bis-guanidinium-spermidine-cholesterol (BGTC); bis-guanidinium-tren-cholesterol (BGTC); 1,3-Di-oleoyloxy-2-(6-carboxy-spermyl)-propylamid (DOSPER) N-[3-[2-(1,3-dioleoyloxy)propoxy-carbonyl]propyl]-N,N,N-trimethylammonium iodide (YKS-220); as well as pharmaceutically acceptable salts and mixtures thereof. Additional examples of cationic lipids are described in Lasic and Papahadjopoulos, Medical Applications of Liposomes, Elsevier, 1998; U.S. Pat. Nos. 4,897,355; 5,208,036; 5,264,618; 5,279,833; 5,283,185; 5,334,761; 5,459,127; 5,736,392; 5,753,613; 5,785,992; 6,376,248; 6,586,410; 6,733,777; and 7,145,039.

Lipid-based carriers included in compositions, kits and methods of the invention also include non-cationic lipids, such as neutral, zwitterionic, and anionic lipids. Examples of non-cationic lipids include, but are not limited to, dioleoyl phosphatidylethanolamine (DOPE); and 1,2-dioleoyl-sn-glycero-3-Phosphocholine (DOPC).

Suitable mixtures of lipids which are lipid-based carriers are well-known in the art and include, but are not limited to: a 3:1 (w/w) formulation of DOSPA and DOPE, under the trade name Lipofectamine™ (Invitrogen); a 1:1 w/w formulation of DOTMA and DOPE is available under the trade name Lipofectin® (Invitrogen); and mixtures of DOTMA and DOPE.

A lipid-based carrier is formulated as a liposome for use in compositions, kits and methods according to embodiments of the invention. The term “liposome” refers to a bilayer particle of amphipathic lipid molecules enclosing an aqueous interior space. Liposomes are typically produced as small unilamellar vesicles (SUVs), large unilamellar vesicles (LUVs) or multilamellar vesicles (MLVs). A material to be delivered to a cell is complexed with liposomes by encapsulation in the aqueous interior space of the liposomes, disposed in the lipid bilayer of the liposomes and/or associated with the liposomes by binding, such as ionic binding or association by van der Waals forces.

Liposomes can be synthesized by methods well-known in the art or obtained commercially. Liposomes included in compositions, kits and methods according to embodiments of the invention are generally in the range of about 1 nanometer-1 micron in diameter although they are not limited with regard to size.

Liposomes are generated using well-known standard methods, including, but not limited to, solvent/hydration methods, ethanol or ether injection methods, freeze/thaw methods, sonication methods, reverse-phase evaporation methods, and surfactant methods. Liposomes and methods relating to their preparation and use are found in Liposomes: A Practical Approach (The Practical Approach Series, 264), V. P. Torchilin and V. Weissig (Eds.), Oxford University Press; 2nd ed., 2003; N. Duzgunes, Liposomes, Part A, Volume 367 (Methods in Enzymology) Academic Press; 1st ed., 2003; L. V. Allen, Jr. et al., Ansel's Pharmaceutical Dosage Forms and Drug Delivery Systems, 8th Ed., Philadelphia, Pa.: Lippincott, Williams & Wilkins, 2005, pp. 663-666; and A. R. Gennaro, Remington: The Science and Practice of Pharmacy, Lippincott Williams & Wilkins, 21st ed., 2005, pp. 766-767. Commercially available liposome preparations used for lipofection include Lipofectamine™ and Lipofectamine™ 2000.

A material included in a lipid-based carrier complex for delivery to cells is a nucleic acid according to embodiments of compositions, kits and methods of the present invention.

The terms “nucleic acid” refers to oligomeric or polymeric molecules containing at least two deoxynucleotides or ribonucleotides and can be linear or circular, single-stranded or double-stranded, encompassing oligonucleotides and polynucleotides. Nucleic acids include DNA and RNA, such as, but not limited to, plasmid DNA, cDNA, mRNA, tRNA, rRNA, mtDNA, siRNA, shRNA, ribozymes, BAC DNA, artificial chromosomes, chromosomal DNA, cosmids, fosmids, expression cassettes and antisense oligonucleotides. The nucleic acid can be derived from any of various sources including but not limited to, bacterial, yeast, plant, bird, insect, fish, and mammals, such as human, non-human primate, mouse, rat, guinea pig, horse, cow, pig, sheep, goat and rabbit. Chimeric nucleic acids, derived from two or more sources, can be used.

Naturally occurring nucleotides or ribonucleotides can be included in nucleic acids to be delivered into cells according to methods of the present invention. Nucleotide or ribonucleotide analogs can be included in nucleic acids delivered into cells according to methods of the present invention. The terms “nucleotide analog” and “ribonucleotide analog” refer to a modified or non-naturally occurring nucleotide or ribonucleotide which can be polymerized, with or without naturally occurring nucleotides or ribonucleotides and incorporated into a nucleic acid. Nucleotide and ribonucleotide analogs are well-known in the art. Particular nucleotide or ribonucleotide analogs include, but are not limited to, those containing an analog of a nucleotide or ribonucleotide base such as substituted purines or pyrimidines, deazapurines, methylpurines, methylpyrimidines, aminopurines, aminopyrimidines, thiopurines, thiopyrimidines, indoles, pyrroles, 7-deazaguanine, 7-deazaadenine, 7-methylguanine, hypoxanthine, pseudocytosine, pseudoisocytosine, isocytosine, isoguanine, 2-thiopyrimidines, 4-thiothymine, 6-thioguanine, nitropyrrole, nitroindole, and 4-methylindole. Nucleotide analogs include those containing an analog of a deoxyribose such as a substituted deoxyribose, a substituted or non-substituted arabinose, a substituted or non-substituted xylose, and a substituted or non-substituted pyranose. Nucleotide and ribonucleotide analogs include those containing an analog of a phosphate ester such as phosphorothioates, phosphorodithioates, phosphoroamidates, phosphoroselenoates, phosophoroanilothioates, phosphoroanilidates, phosphoroamidates, boronophosphates, phosphotriesters, and alkylphosphonates such as methylphosphonates.

A nucleic acid to be delivered into cells according to methods of the present invention can be in any of a variety of forms including, but not limited to, expression vectors, such as cDNA plasmids and viruses; mRNA; and inhibitory RNA.

The term “protein” refers to a chain of amino acids linked by peptide bonds. The term protein encompasses oligopeptides and polypeptides.

The term “inhibitory RNA” refers to RNA molecules active to specifically decrease levels or function of a target RNA in cells, such as by RNA interference. Inhibitory RNA includes antisense RNA, RNAi, shRNA, siRNA and micro RNA (miRNA).

RNA interference is a target sequence-specific method of inhibiting a selected gene. RNA interference has been characterized in numerous organisms and is known to be mediated by a double-stranded RNA, also termed herein a double-stranded RNA compound. Briefly described, RNA interference involves a mechanism triggered by the presence of small interfering RNA, siRNA, resulting in degradation of a target complementary mRNA. siRNA is double-stranded RNA which includes a nucleic acid sequence complementary to a target sequence in the gene to be silenced. The double-stranded RNA may be provided as a long double-stranded RNA compound, in which case it is subject to cleavage by the endogenous endonuclease Dicer in a cell. Cleavage by Dicer results in siRNA duplexes having about 21-23 complementary nucleotides in each of the sense strand and the antisense strand, and optionally 1-2 nucleotide 3′ overhangs on each of the two strands.

Alternatively, siRNA is provided as a duplex nucleic acid having a sense strand and an antisense strand, wherein the sense and antisense strands are substantially complementary and each of the sense and antisense strands have about 16-30 nucleotides. The complementary sense and antisense strands and optionally include 1-2 nucleotide 3′ overhangs on one or both of the two strands. In one embodiment, a siRNA is preferred which has sense and antisense strands, wherein each of the two strands has 21-23 nucleotides, wherein 2 nucleotides on the 3′ end of each strand are overhanging and the remaining 19-21 nucleotides are 100% complementary. As noted above, further details of siRNA compounds are described in Engelke, D. R., RNA Interference (RNAi): Nuts and Bolts of RNAi Technology, DNA Press LLC, Eagleville, Pa., 2003. Additional description of siRNA length and composition is found in Elbashir, S. M. et al., Genes and Dev., 15:188-200, 2001; and O'Toole, A. S. et al., RNA, 11:512-516, 2005.

siRNA provided as a duplex nucleic acid having a sense strand and an antisense strand may be configured such that the sense strand and antisense strand form a duplex in hybridization conditions but are otherwise unconnected. A double-stranded siRNA compound may be assembled from separate antisense and sense strands. Thus, for example, complementary sense and antisense strands are chemically synthesized and subsequently annealed by hybridization to produce a synthetic double-stranded siRNA compound.

Further, the sense and antisense strands for inclusion in siRNA may be produced from one or more expression cassettes encoding the sense and antisense strands. Where the sense and antisense strands are encoded by a single expression cassette, they may be excised from a produced transcript to produce separated sense and antisense strands and then hybridized to form a duplex siRNA. See, for example, Engelke, D. R., RNA Interference (RNAi): Nuts and Bolts of RNAi Technology, particularly chapters 5 and 6, DNA Press LLC, Eagleville, Pa., 2003 for further details of synthetic and recombinant methods of producing siRNA.

In a further alternative, a double-stranded “short hairpin” RNA compound, termed “shRNA” or “hairpin siRNA” includes an antisense strand and a sense strand connected by a linker. shRNA may be chemically synthesized or formed by transcription of a single-stranded RNA from an expression cassette in a recombinant nucleic acid construct. The shRNA has complementary regions which form a duplex under hybridization conditions, forming a “hairpin” conformation wherein the complementary sense and antisense strands are linked, such as by a nucleotide sequence of about 1-20 nucleotides. In general, each of the complementary sense and antisense strands have about 16-30 nucleotides.

As noted, siRNA and shRNA may be expressed from a DNA template encoding the desired transcript or transcripts. A DNA template encoding the desired transcript or transcripts is inserted in a vector, such as a plasmid or viral vector, and operably linked to a promoter for expression in vitro or in vivo.

As will be recognized by one of skill in the art, particular siRNAs may be of different size and still be effective to inhibit a target gene. Routine assay may be performed to determine effective size and composition of particular compounds. Without wishing to be bound by theory, it is believed that at least the antisense strand is incorporated into an endonuclease complex which cleaves the target mRNA complementary to the antisense strand of the siRNA.

Administration of long RNA duplexes processed to siRNA, as well as administration of siRNA or shRNA, and/or expression constructs encoding siRNA or shRNA, results in degradation of the target mRNA and inhibition of expression of the protein encoded by the target mRNA, thereby inhibiting activity of the encoded protein in the cell.

Further details of RNA interference mechanisms as well as descriptions of target identification, synthetic siRNA and shRNA production, siRNA and shRNA expression construct production, and protocols for purification and delivery of expression constructs and synthetic siRNA and shRNA in vitro and in vivo are described in Engelke, D. R., RNA Interference (RNAi): Nuts and Bolts of RNAi Technology, DNA Press LLC, Eagleville, Pa., 2003.

Compositions including a lipid-based carrier, a material to be introduced into a cell, and BDM are provided according to the present invention. The compositions can include the lipid-based carrier, material to be introduced into a cell, and BDM in a carrier, such as a cell compatible buffer. The composition can be diluted for application to cells, or can be provided at “ready to use” concentrations for application to cells.

A cell-compatible buffer is any of various buffers which are not toxic to cells, such as, but not limited to, DMEM.

Kits for use in promoting material transfer into a cell according to embodiments of the present invention include an electroporation reagent; 2,3-butanedione monoxime; and a lipofection reagent. Included electroporation reagents are exemplified by cell-compatible buffers. Included lipofection reagents are exemplified by liposomes, cationic liposomes, cationic lipids and non-cationic lipids.

Embodiments of inventive compositions and methods are illustrated in the following examples. These examples are provided for illustrative purposes and are not considered limitations on the scope of inventive compositions and methods.

### EXAMPLES

### Example 1

Nucleic acids are introduced into stellate ganglion (SG) neurons by transfecting Cy3-tagged siRNA using methods and compositions according to embodiments of the present invention. siRNA physically linked to the red fluorophore Cy3 is commercially available from Ambion, Inc., USA.

Adult rat SG neurons from male Wistar rats are employed. The rats are sacrificed by CO₂ anesthesia and decapitated using a laboratory guillotine. The SG tissue is removed and cleared of connective tissue in ice-cold Hanks' balanced salt solution. Thereafter, the SG tissue is placed in a 2 milliliter microcentrifuge tube containing Opti-MEM® (Invitrogen, Inc., USA) supplemented with BDM (2 millimolar, Sigma Chem. Co., USA) and the tube is placed on ice. Two transfection solutions are prepared: Solution A and Solution B.

Solution A is prepared for use with a 12-well plate. The following are gently mixed in a 2 milliliter RNase- and DNase-free microcentrifuge tube: 860 microliters Opti-MEM®, 10 microliters of Lipofectamine 2000 (Invitrogen, Inc., USA), 20 microliters of BDM (0.1 Molar stock, final concentration is 2 millimolar in 1 milliliter) and 10 microliters of Cy3-tagged siRNA (50 micromolar stock, final concentration is 500 nanomolar in 1 milliliter). For certain control experiments, Solution A is made without siRNA. The total final volume of Solution A is 900 microliters. Once Solution A is thoroughly mixed, it is stored in a humidified incubator (5% CO₂/195% air) at 37° C. for 30 minutes.

Solution B is prepared by gently mixing 88 microliters of R solution (provided with the Neon electroporation kit, Invitrogen, USA), 2 microliters of BDM (0.1 Molar stock, final concentration is 2 millimolar in 100 microliters) and 10 microliters of Cy3-tagged siRNA (5 micromolar stock, final concentration is 500 nanomolar in 100 microliters) in a 2 milliliter RNase- and DNase-free microcentrifuge tube. In this example, the final volume for Solution B is 100 microliters. The volume of Solution B prepared is generally equal to the volume of the pipette tip chamber used with the Neon electroporation system, Invitrogen, USA.

The Opti-MEM®/BDM solution is removed from the tube containing the isolated SG tissue and 100 microliters of Solution B is then transferred to this tube. The SG tissue is incubated in Solution B for about 20-25 minutes at room temperature prior to electroporation of the neurons. The tube is gently agitated every 2-3 minutes to allow Solution B to mix with the tissue.

Solution B and SG tissue are drawn up to the 100 microliter electroporation tip and the tip is then inserted into the Neon electroporator. The SG tissue is electroporated at 1000 Volts, with a 20 millisecond duration and applied once (1 pulse).

After electroporating the tissue, Solution B and SG tissue are pipetted into 900 microliters Solution A in a 12-well plate which is placed in a humidified incubator (5% CO₂/95% air) at 37° C. for 4-6 hrs.

Liquid is then removed from the electroporated SG tissue which is rinsed 3 times with minimal essential medium (MEM), supplemented with 10% fetal calf serum, 1% glutamine and 1% penicillin-streptomycin (all from Invitrogen, Inc, USA). After the third rinse, the electroporated SG tissue is incubated in 1 milliliter of the supplemented MEM in a humidified incubator (5% CO₂/95% air) at 37° C. overnight.

The following day, the electroporated SG tissue is enzymatically dissociated as described in J. Neurophys., 2008, 100:1420-32 and the dispersed SG neurons are then allowed to attach to the bottom of 35 mm dishes for about 3 to 6 hours. Thereafter, phase and fluorescence imaging is used to image the dispersed neurons and analyze transfection efficiency. This analysis shows that greater than 90% of the neurons are transfected with Cy3-tagged siRNA.

### Example 2

Introduction of nucleic acids into cells using the method and compositions described in Example 1 was compared with alternate methods and compositions in this example. Variations compared include 1) electroporation of SG tissue with 500 nanomolar Cy3-tagged siRNA, in the presence of 2 millimolar BDM, without Lipofectamine™ treatment; 2) electroporation of SG tissue with Cy3-tagged siRNA in the presence of 2 millimolar BDM followed by incubation of the electroporated tissue with Lipofectamine™ 2000 treated 500 nanomolar Cy3-tagged siRNA in the presence of 2 millimolar BDM for various times; and 3) electroporation of SG tissue without siRNA in the presence of 2 millimolar BDM followed by transfection of the SG tissue with Lipofectamine™ 2000 treated 500 nanomolar Cy3-tagged siRNA in the presence of 2 millimolar BDM. Phase and fluorescence imaging is used to image the SG neurons and analyze transfection efficiency. This analysis unexpectedly shows greater transfection using electroporation in combination with lipofection in the presence of BDM.

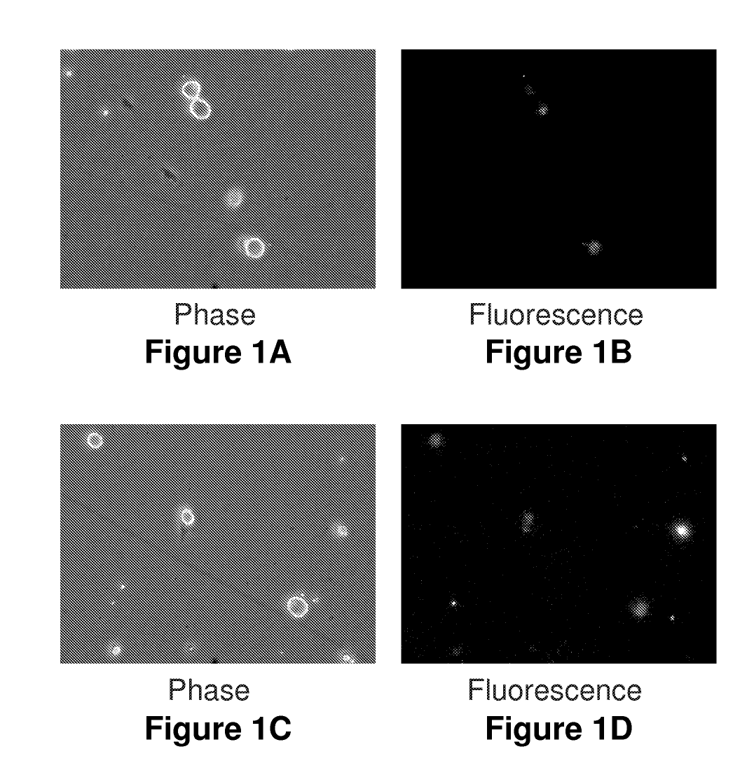

FIG. 1 illustrates results of transfection by electroporation of SG tissue with 500 nanomolar Cy3-tagged siRNA in the presence of 2 millimolar BDM without Lipofectamine™. FIG. 1 shows the fluorescence and phase images at 10× magnification of the dissociated SG neurons. Only weak fluorescence is detectable in a few cells using an 800 ms exposure with suitable excitation and emission filters.

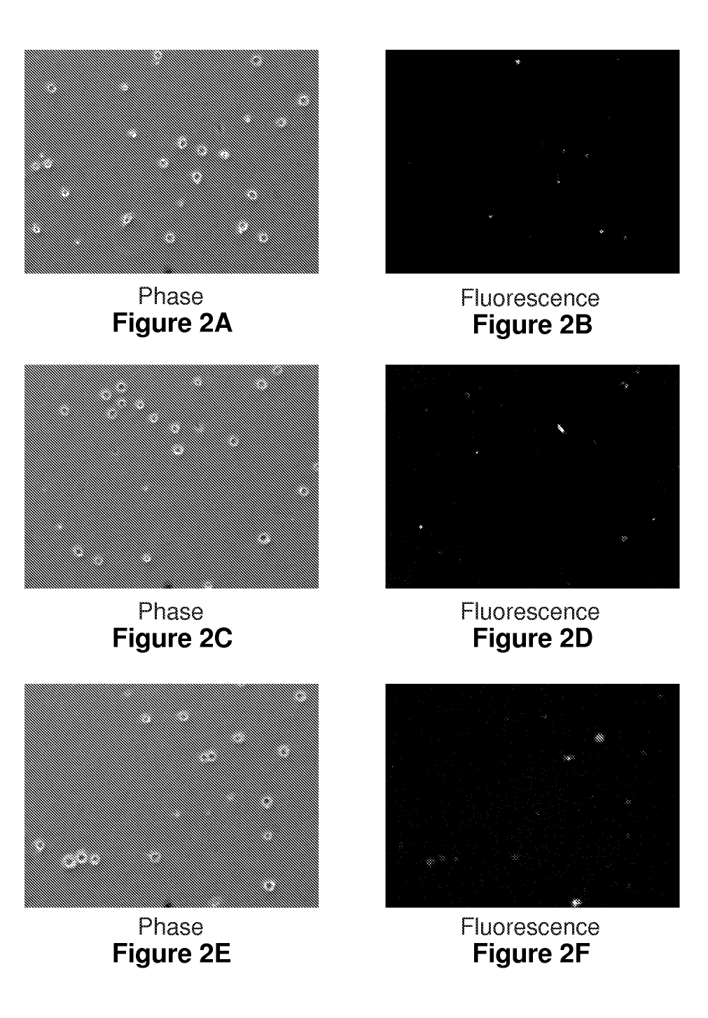

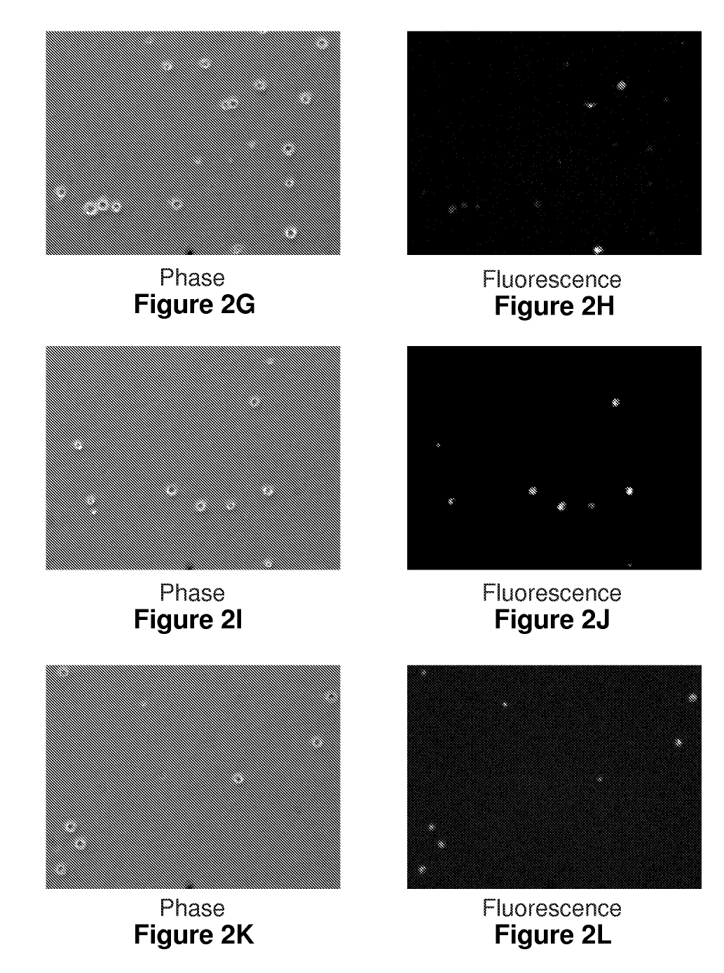

FIG. 2 illustrates comparison of 1) electroporation of SG tissue with 500 nanomolar Cy3-tagged siRNA in the presence of 2 millimolar BDM; and 2) electroporation of SG tissue with Cy3-tagged siRNA in the presence of 2 millimolar BDM followed by lipofection with Lipofectamine™ 2000 complexed with 500 nanomolar Cy3-tagged siRNA in the presence of 2 millimolar BDM. FIG. 2 shows the fluorescence images alongside phase images at 10× magnification of the dissociated SG neurons.

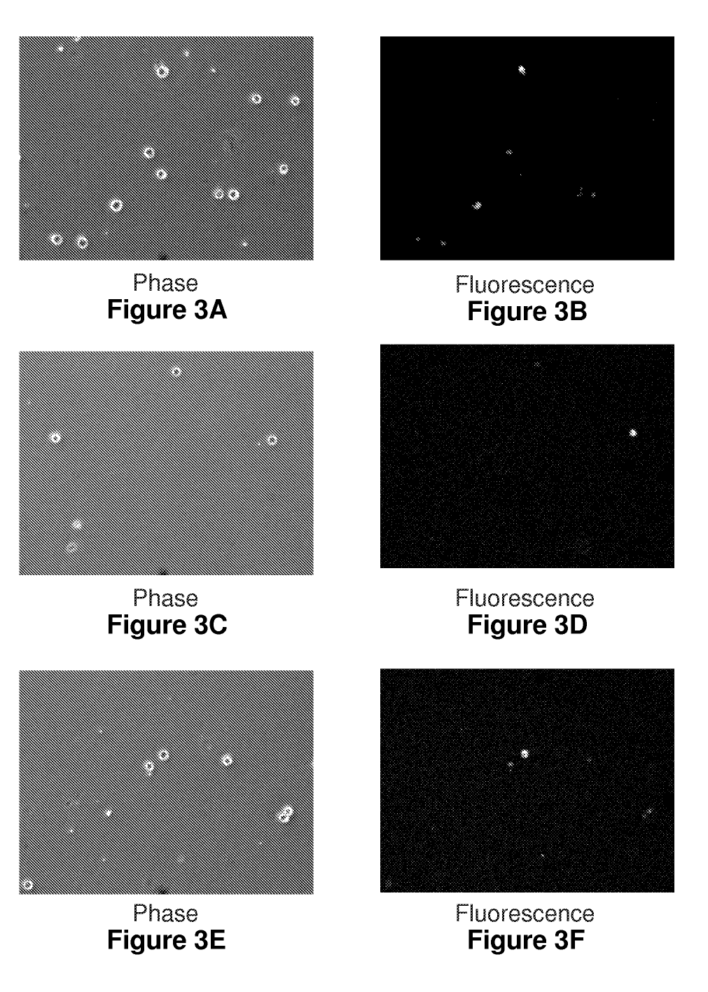

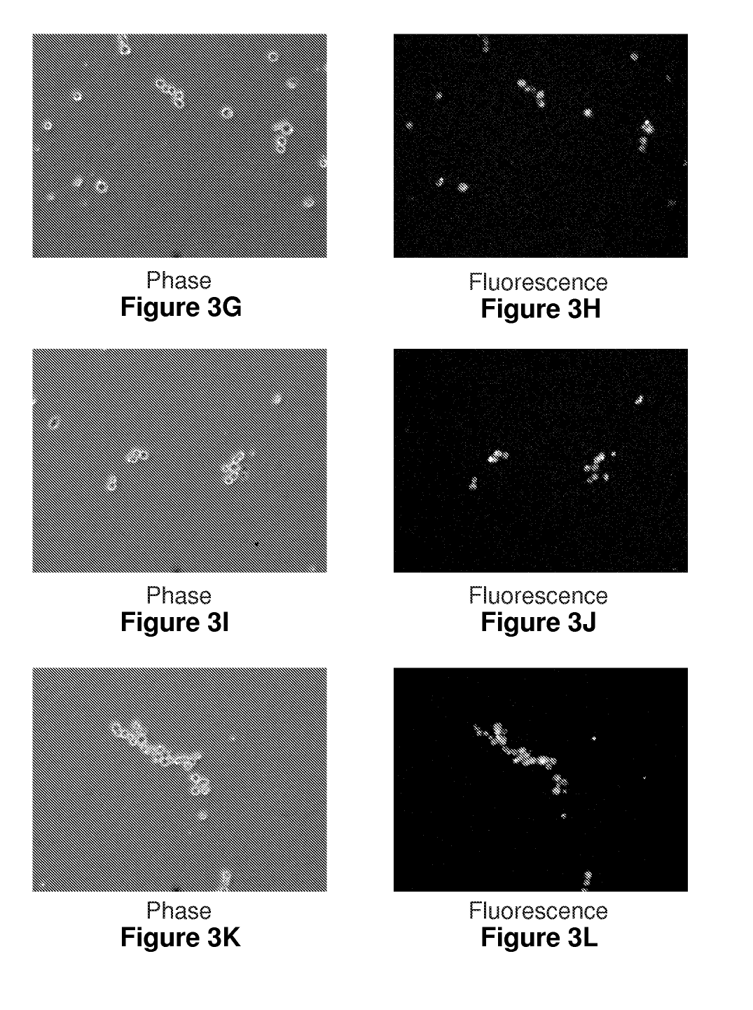

FIG. 3 illustrates comparison of 1) electroporation of SG tissue with Cy3-tagged siRNA in the presence of 2 millimolar BDM followed by incubation with Lipofectamine™ 2000 complexed with 500 nanomolar Cy3-tagged siRNA for one hour in the presence of 2 millimolar BDM; and 2) electroporation of SG tissue with Cy3-tagged siRNA in the presence of 2 millimolar BDM followed by incubation with Lipofectamine™ 2000 treated 500 nanomolar Cy3-tagged siRNA for two hours in the presence of 2 millimolar BDM. FIG. 3 shows the fluorescence images alongside phase images at 10× magnification of the dissociated SG neurons. More highly fluorescent neurons are found following longer incubation of the electroporated tissue with Lipofectamine™ 2000 treated 500 nanomolar Cy3-tagged siRNA.

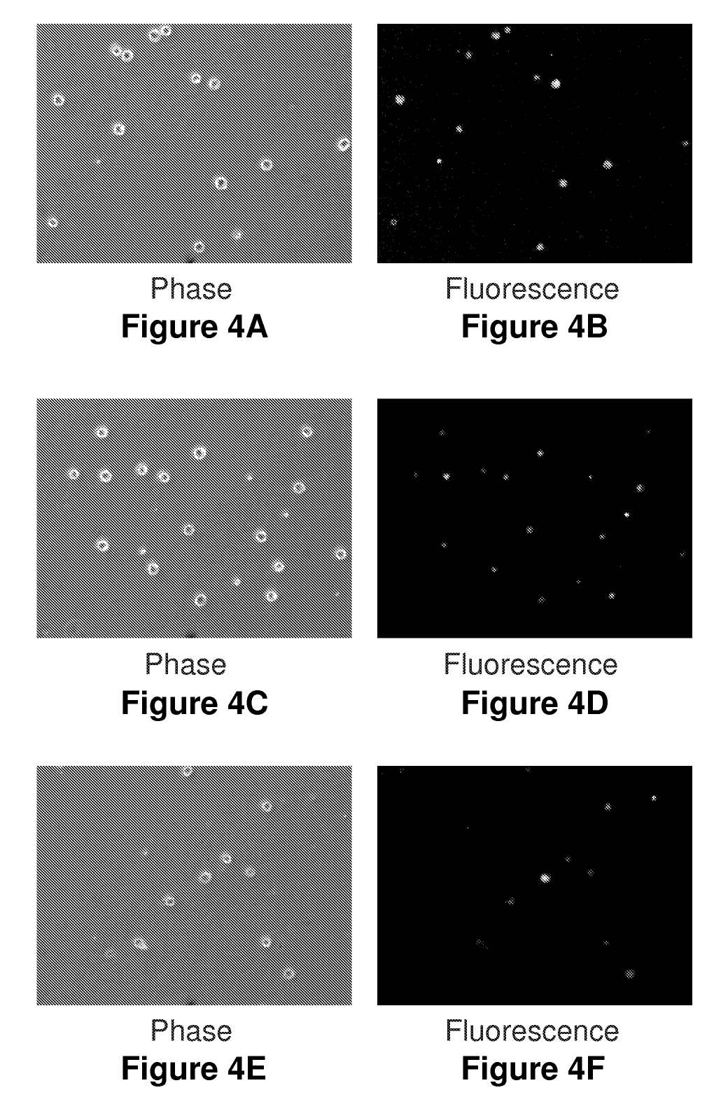

FIG. 4 illustrates electroporation of SG tissue without siRNA in the presence of 2 millimolar BDM followed by transfection of the SG tissue with Lipofectamine™ 2000 treated 500 nanomolar Cy3-tagged siRNA in the presence of 2 millimolar BDM. Phase and fluorescence imaging is used to image the SG neurons and analyze transfection efficiency. FIG. 4 shows the fluorescence images and phase images at 10× magnification of the dissociated SG neurons.

The transfection efficiency was greater than 90% for all 3 groups and the efficiency of transfection with the combination of electroporation in the presence of BDM and lipofection in the presence of BDM was greater than 95%. Immunofluorescence studies revealed a brighter fluorescence signal for Cy3-labeled siRNA in SG neurons when the tissue was transfected simultaneously with both electroporation and lipofection techniques.

### Example 3

DNA is introduced into the MCF-7 human breast cancer cell line by transfecting a cDNA vector encoding a green fluorescent protein used as a marker for analysis of transfection.

Three transfection solutions are prepared and referred to as Solution A, Solution B and Solution C.

Solution A is prepared for use with a 6-well plate. The following are gently mixed in a 15 milliliter RNase- and DNase-free centrifuge tube: 2940 microliters Opti-MEM®, 60 microliters of Lipofectamine™ 2000 (Invitrogen, Inc., USA). Once Solution A is thoroughly mixed, it is allowed to stand at room temperature for 30 minutes.

Solution B is prepared by gently mixing 2826 microliters of Opti-MEM® and 54 microliters of cDNA vector (1 microgram/microliter stock concentration) obtained from Genscript. In this example, each well will be transfected with 9 micrograms cDNA. As with Solution A above, Solution B is allowed to stand at room temperature for 30 minutes.

Solution C is prepared by gently mixing 534 microliters of Opti-MEM® and 54 microliters of cDNA vector (1 microgram/microliter stock concentration).

Following the 30 minute period, Solution A is filtered through a 0.8 micron Supor® Membrane low protein binding Acrodisc syringe filter (Pall Life Sciences), and the filtered Solution A is allowed to drip onto a new 15 milliliter RNase- and DNase-free centrifuge tube. Thereafter, both Solution A and Solution B are gently mixed together (final volume is ˜5880 microliters) and allowed to stand are room temperature for 30 minutes (now called Solution A/B). At this point, the MCF-7 breast cancer cells are plated onto the 6-well plate.

For this example, it is assumed that the MCF-7 cells are grown in a 75 cm³ flask with DMEM media supplemented with 10% fetal calf serum, 1% glutamine and 1% penicillin-streptomycin (supplemented DMEM) and that the total number of MCF-7 cells in the flask is approximately 10 million. In general, 10-15 million cells per 75 cm³ flask is an optimum number for transfection. It is difficult to transfect cells that are quite confluent (i.e. greater than 20 million cells) min flasks.

Prior to plating the cells, the following solutions are warmed to 37° C.: 1× Dulbecco's Phosphate Buffered Saline (DPBS) with Ca²⁺ and Mg²⁺, 1×DPBS (without Ca²⁺ and Mg²⁺), Opti-MEM®, trypsin (0/05%), and supplemented DMEM (described above).

First, the DMEM is removed from the flask containing the MCF-7 cells and is rinsed once with 10 milliliters of warm 1×DPBS (without Ca²⁺ and Mg²⁺). Thereafter the DPBS is aspirated and 4 milliliters of trypsin (0.05%) are added to the flask. The flask is placed back in the humidified incubator (5% CO₂/95% air) for 120-180 seconds.

At the end of the 120 second period, the flask is tapped against a surface so as to maximize the detachment of cells that are still attached to the bottom of the flask. Thereafter, 7 milliliters of supplemented DMEM is added to the flask and the cells are transferred to a 15 milliliter Ambion tube and spun for 5 minutes at 900 rpm. In general, cells are not exposed to trypsin alone for more than 3 minutes.

After centrifugation, the supernatant is removed and the cell pellet is gently broken up with 10 milliliters of warm DPBS (with Ca²⁺ and Mg²⁺). A small volume (˜100 microliters) of the 10 milliliter cell suspension is removed to count the cells employing the Cell Countess (Invitrogen).

It is assumed that the cell count was 10 million total live cells in a 10 milliliter suspension, such that cell concentration is 1 million cells/milliliter. In a 6-well plate, 300,000 cells/dish are plated and so a total of 1.8 million cells are required. Thus, 1.8 milliliters of the cell suspension is placed in a new 15 milliliter Ambion tube and spin again for 5 minutes at 900 rpm.

After centrifugation, as much of the supernatant is removed as possible and a pellet of cells remains (˜1.8 million).

Steps described above typically take 30 minutes or less to execute. Thus, just as the cells have been spun down, 30 minutes have elapsed. At this point, 120 microliters of BDM (0.1 Molar stock) are added to Solution A/B and 12 microliters of BDM (0.1 Molar stock) are added to Solution C, both are gently mixed.

After mixing the solutions, 900 microliters of Solution A/B are added to each of the wells in the 6-well plate, and the plate is placed in the humidified incubator (5% CO₂/95% air).

Solution C is then used to gently break up the cell pellet (˜1.8 million cells) that was spun previously in a 15 milliliter centrifuge tube. Once the pellet is broken up, the cells are transferred to a 2 milliliter Ambion tube and stored at room temperature for 15 to 20 minutes. This incubation period allows for the cDNA to come in close contact with the cells. Every 2 minutes, the 2 milliliter Ambion tube is gently flicked so that the cells are not allowed to settle down to the bottom of the tube. This remixing of the tube is necessary so that the cDNA can reach the surface of all the cells. If the cells are allowed to settle (easy to visualize with the naked eye), transfection (via electroporation) is not optimal.

Following the 20 minute incubation of the MCF-7 cells in Solution C, the cells are drawn up to the 100 microliters electroporation tip and the tip is then inserted into the Neon electroporator. The cells are electroporated at 1250 Volts, with a 10 millisecond duration applied three times (3 pulses). The cells are then transferred to one of the 6 wells in the plate. This process is repeated 5 more times until all 6 dishes have had electroporated cells placed in the plate. The 6-well plate is then placed in the humidified incubator (5% CO₂/95% air) at 37° C. for 6 hours.

After the 6 hour incubation of the cells, each dish is rinsed 3 times with the supplemented DMEM and then they are replenished with supplemented DMEM and returned to the humidified incubator (5% CO₂/95% air).

After overnight incubation, the efficiency of cDNA transfection can be observed with the use of a microscope with fluorescence filter optimized for the green fluorescent protein. The analysis shows more than 90% transfection efficiency.

### Example 4

DNA is introduced into cells of the MTR5 human breast cancer cell line by transfecting a cDNA vector encoding a green fluorescent protein used as a marker for analysis of transfection.

MTR5 cells are similar to the MCF-7 cells, except that they are resistant to Tamoxifen-mediated death.

The procedure described in Example 3 is used and efficiency of transfection is analysed by fluorescence microscopy, showing more than 90% transfection efficiency.

### Example 5

DNA is introduced into cells of the U87-MG human glioma cell line by transfecting a cDNA vector encoding a green fluorescent protein used as a marker for analysis of transfection.

The procedure described in Example 3 is used with modification of the electroporation step such that the cells are electroporated at 1350 Volts, with a 14 millisecond duration applied three times (3 pulses) and efficiency of transfection is analysed by fluorescence microscopy, showing more than 90% transfection efficiency.

### Example 6

DNA is introduced into cells of the U251 human glioma cell line by transfecting a cDNA vector encoding a green fluorescent protein used as a marker for analysis of transfection.

The procedure described in Example 3 is used with modification of the electroporation step such that the cells are electroporated at 1350 Volts, with a 14 millisecond duration applied three times (3 pulses) and efficiency of transfection is analysed by fluorescence microscopy, showing more than 90% transfection efficiency.

### Example 7

Stellate Ganglion and Superior Cervical Ganglion Tissue and Cell Isolation

Whole stellate ganglion (SG) and superior cervical ganglion (SCG) tissue from adult rats are removed and cleared of connective in ice-cold Hanks' balanced salt solution. Thereafter, the tissue is placed in ice-cold Opti-MEM® (Invitrogen) until ready for siRNA transfection.

siRNA Transfection

SG or SCG neurons are transfected with Cy3-labeled siRNA via lipofection alone for comparison with other techniques. The SG or SCG tissue is transferred to a 22 mm dish containing Opti-MEM® supplemented with 10 microliters of Lipofectamine™ 2000 (Invitrogen), Cy3-labeled siRNA (500 nanomolar, Ambion), 2,3-butanedione monoxime (BDM, 2 millimolar), final volume of 1000 microliters. The SG tissue is incubated in this solution for 4 hours in a humidified incubation (5% CO₂/95% air) at 37° C. After the 4 hour period, SG or SCG tissue is dissociated in Earle's Balanced Salt Solution containing 0.6 milligrams/milliliter collagenase and 0.4 milligrams/milliliter trypsin at 35° C. The dispersed neurons are stored in MEM (10% PBS, 1% glutamine and 1% Pen-Strep) overnight. The following day, images of the cells are obtained with a Nikon TE2000 microscope and acquired with an Orca-ER 1394 digital CCD camera (Hamamatsu) and iVision software (Biovision).

Some SG or SCG neurons are transfected with Cy3-labeled siRNA via electroporation alone for comparison with other techniques. Electroporation is carried out with the NEON™ Electroporator (Invitrogen) by preincubating for 15-20 minutes the tissue in an RNase- and DNase-free microcentrifuge tube containing the R solution (provided with the NEON™ Kit), Cy3-labeled siRNA (500 nanomolar) and BDM (2 millimolar) with a final volume of 100 microliters. Afterwards, the SG tissue is drawn up to the 100 microliter electroporator pipet tip and electroporated with a single 20 msec 1000 Volt pulse. The SG or SCG tissue is then dissociated in Earle's Balanced Salt Solution containing 0.6 milligrams/milliliter collagenase and 0.4 milligrams/milliliter trypsin at 35° C. The dispersed neurons are stored in MEM (10% FBS, 1% glutamine and 1% Pen-Strep) overnight. The following day, images of the cells are obtained with a Nikon TE2000 microscope and acquired with an Orca-ER 1394 digital CCD camera (Hamamatsu) and iVision software (Biovision).

When both transfection techniques are employed, electroporation of Cy3-labeled siRNA is carried out first and followed by lipofection. Electroporation is carried out with the NEON™ Electroporator (Invitrogen) by preincubating for 15-20 minutes the tissue in an RNase- and RNase-free microcentrifuge tube containing the R solution (provided with the NEON Kit), Cy3-labeled siRNA (500 nanomolar) and BDM (2 millimolar) with a final volume of 100 microliters. Afterwards, the SG tissue is drawn up to the 100 microliter electroporator pipet tip and electroporated with a single 20 millisecond 1000 Volt pulse. The SG or SCG tissue is transferred to a 22 mm dish containing Opti-MEM® supplemented with 10 microliters of Lipofectamine 2000 (Invitrogen), Cy3-labeled siRNA (500 nanomolar, Ambion), 2,3-butanedione monoxime (BDM, 2 millimolar), final volume of 1000 microliters. The SG tissue is incubated in this solution for 4 hr in a humidified incubation (5% CO₂/95% air) at 37° C. After the 4 hour period, the SG or SCG tissue is dissociated in Earle's Balanced Salt Solution containing 0.6 milligrams/milliliter collagenase and 0.4 milligrams/milliliter trypsin at 35° C. The dispersed neurons are stored in MEM (10% FBS, 1% glutamine and 1% Pen-Strep) overnight. The following day, images of the cells are obtained with a Nikon TE2000 microscope and acquired with an Orca-ER 1394 digital CCD camera (Hamamatsu) and iVision software (Biovision).

siRNA oligonucleotides directed against the nociceptin/orphanin FQ peptide (NOP) receptor expressed in SG neurons are transfected into SG neurons using methods of the present invention in this example. Electrophysiological, molecular and biochemical techniques are used to monitor the expression pattern of the NOP receptor in transfected cells following siRNA transfection.

Electroporation of two siRNA sequences to silence the NOP receptors: TCATTGCTATCGACTACTA (SEQ ID NO. 1) and AGACAGCTACCAACATTTA (SEQ ID NO. 2) is carried out first, followed by lipofection using the same sequences. Electroporation is carried out with the NEON™ Electroporator (Invitrogen) by preincubating for 15-20 min the SG tissue in an RNase- and DNase-free microcentrifuge tube containing the R solution (provided with the NEON Kit), anti-NOP siRNA (500-1500 nanomolar) and BDM (2 millimolar) with a final volume of 100 microliters. Afterwards, the SG tissue is drawn up to the 100 microliter electroporator pipet tip and electroporated with a single 20 msec 1000 Volt pulse. The SG or SCG tissue is transferred to a 22 mm dish containing Opti-MEM® supplemented with 10 microliters of Lipofectamine™ 2000 (Invitrogen), anti-NOP siRNA (500-1500 nanomolar), 2,3-butanedione monoxime (BDM, 2 millimolar), final volume of 1000 microliters. The SG tissue is incubated in this solution for 4 hr in a humidified incubation (5% CO₂/95% air) at 37° C. After the 4 hour period, the SG tissue is stored in MEM (10% FBS, 1% glutamine and 1% Pen-Strep) overnight. On day 2 both the lipofection and electroporation are performed again and the SG tissue is either 1) again stored in MEM (10% FBS, 1% glutamine and 1% Pen-Strep) overnight followed by dissociation of the neurons on day 3 and analysis on day 4; or 2) dissociated on day 2 followed by analysis on day 3. The SG tissue is dissociated in Earle's Balanced Salt Solution containing 0.6 milligrams/milliliter collagenase and 0.4 milligrams/milliliter trypsin at 35° C. The dispersed neurons are stored in MEM (10% FBS, 1% glutamine and 1% Pen-Strep) overnight. Images of the cells are obtained with a Nikon TE2000 microscope and acquired with an Orca-ER 1394 digital CCD camera (Hamamatsu) and iVision software (Biovision).

Electrophysiology and Data Analysis

Calcium currents are recorded using the whole cell variant of the patch clamp technique. Whole cell currents are acquired with a patch clamp amplifier, analog filtered at 1-2 kHz (−3 dB; 4 pole Bessel) and digitized using custom designed software (S5) equipped with an 18 bit analog to digital converter board. The external solution contains: TEA-OH 145 millimolar, HEPES 10 millimolar, CaCl₂ 10 millimolar, glucose 15 millimolar and 0.0003 millimolar tetrodotoxin, pH to 7.4 with methanesulfonate. The pipet solution contains: N-methyl-D-glucamine (NMG) methanesulfonate, TEA 20 millimolar, EGTA 11 millimolar, CaCl₂ 1 millimolar, HEPES 10 millimolar, Mg-ATP 4 millimolar, Na₂GTP 0.3 millimolar, Creatine Phosphate 14 millimolar, sucrose 10 millimolar. pH to 7.2 with TEA-OH. Stock solution of Nociceptin (Tocris Cookson) is prepared in water and diluted in the external solution to its final concentration just prior to use.

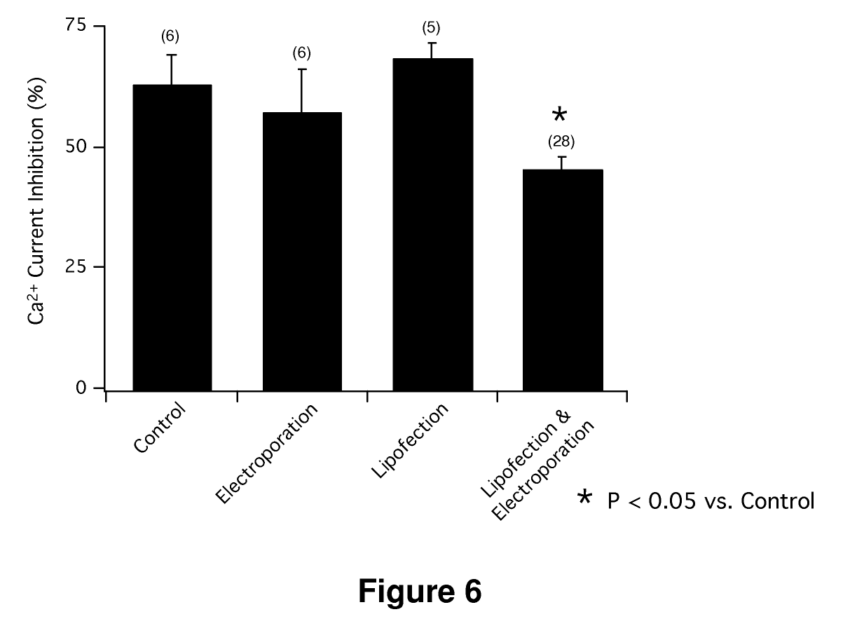

FIG. 6 is a graph showing the effects of transfection of SG neurons in vitro with siRNA oligonucleotides directed against NOP by electroporation+BDM alone, lipofection+BDM alone, or a combination of electroporation+BDM and lipofection+BDM, compared to a control;

Western Blotting

Protein from SG tissue is prepared using a protein lysis buffer (Macherey Nagel). Twenty micrograms of protein are loaded on each lane of NuPAGE 10% Bis-Tris pre-cast gels (Invitrogen). The gels are run at 200 Volts for 55 minutes. Thereafter, the gels are transferred to PVDF membranes. Detection of NOP receptors is performed with rabbit polyclonal antibody to NOP receptors (Abeam) and goat polyclonal antibody to actin. Western blot analysis shows that NOP receptor protein levels decrease 3-4 days post siRNA transfection using a combination of electroporation+BDM and lipofection+BDM.

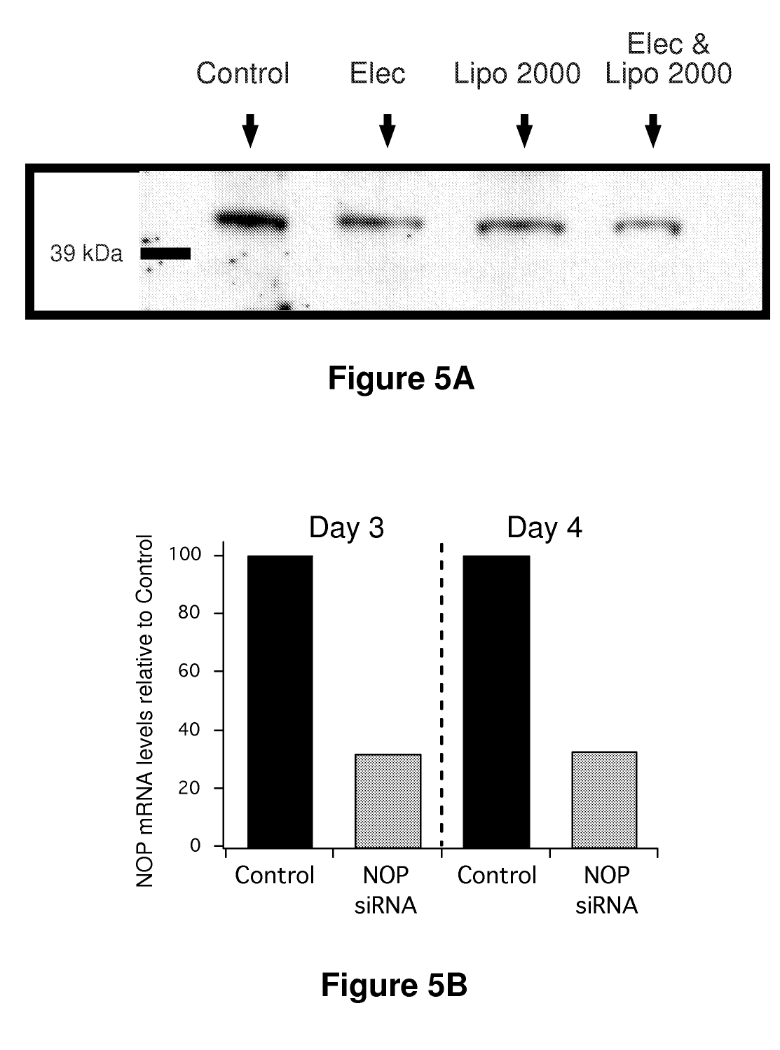

FIG. 5A is an image of a Western blot showing the effects of transfection of SG neurons in vitro with siRNA oligonucleotides directed against the nociceptin/orphanin FQ peptide (NOP) receptor by electroporation+BDM alone, lipofection+BDM alone, or a combination of electroporation+BDM and lipofection+BDM, compared to a control;

Quantitative RT-PCR

SG tissue mRNA is obtained with a lysis buffer (Macherey Nagel). TaqMan® Gene Expression Assays (Applied Biosystems) are employed for measuring rat NOP receptors and GAPDH mRNA levels. The assays are run on a 7900HT PCR System (Applied Biosystems) and analyzed on a PC. Quantitative RT-PCR analysis shows that NOP receptor mRNA levels decrease 3-4 days post siRNA transfection using a combination of electroporation+BDM and lipofection+BDM.

FIG. 5B is a graph showing the effects of transfection of SG neurons in vitro with siRNA oligonucleotides directed against NOP receptors by a combination of electroporation+BDM and lipofection+BDM, compared to a control;

### Example 8

### Stellate Ganglion (SG) Tissue Isolation

Whole SG tissue as well as single neurons from adult rat are employed in this study. Male Wistar rats (175-225 grams) are anaesthetized with CO₂ and then decapitated using a laboratory guillotine. The SG is removed and cleared of connective tissue in ice-cold Hanks' balanced salt solution. Thereafter, the SG tissue is placed in ice-cold Opti-MEM® (Invitrogen) until ready for siRNA transfection.

siRNA Transfection

Transfection of siRNA designed to silence individual Gβ subunits is performed by employing both electroporation+BDM and lipofection+BDM. Electroporation is carried out with the NEON™ Electroporator (Invitrogen) by preincubating for 15-20 minutes the SG tissue in an RNase- and DNase-free microcentrifuge tube containing the R solution (provided with the NEON Kit), Gβ siRNA (1500 nanomolar) and 2,3-butanedione monoxime (BDM, 2 millimolar). Afterwards, the SG tissue is drawn up to the 100 microliter electroporator pipet tip and electroporated with a single 20 millisecond 1000 Volt pulse. The SG tissue is then placed in a 22 mm dish containing Opti-MEM® supplemented with Gβ siRNA (1500 nM), BDM (2 millimolar) and 10 microliters of Lipofectamine™ 2000 (Invitrogen) for 4 hr. Following the 4 hr incubation period, the 22 mm dish is rinsed 3 times with MEM and then stored in MEM supplemented with 2% FBS, 1% Pen-Strep, 1% glutamine. The SG tissue is retransfected with both transfection techniques 48 hr after the initial transfection. The siRNA sequences designed to silence rat Gβ are CAACTGAAGAACCAAATTA (SEQ ID NO. 3) and ACATCTGGGATGCCATGAA (SEQ ID NO. 4), while the sequence used to silence rat Gβ4 is ACAACATCTGCTCCATATA (SEQ ID NO. 5) (all from Ambion). Control groups are transfected with scrambled siRNA sequences (1500 nM, Ambion) also via electroporation+BDM and lipofection+BDM as described for Gβ32 siRNA.

Electrophysiology and Data Analysis

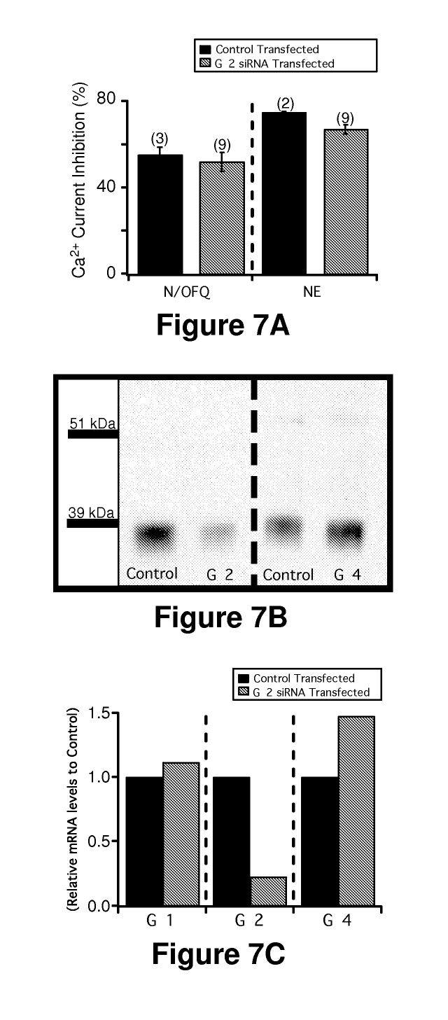

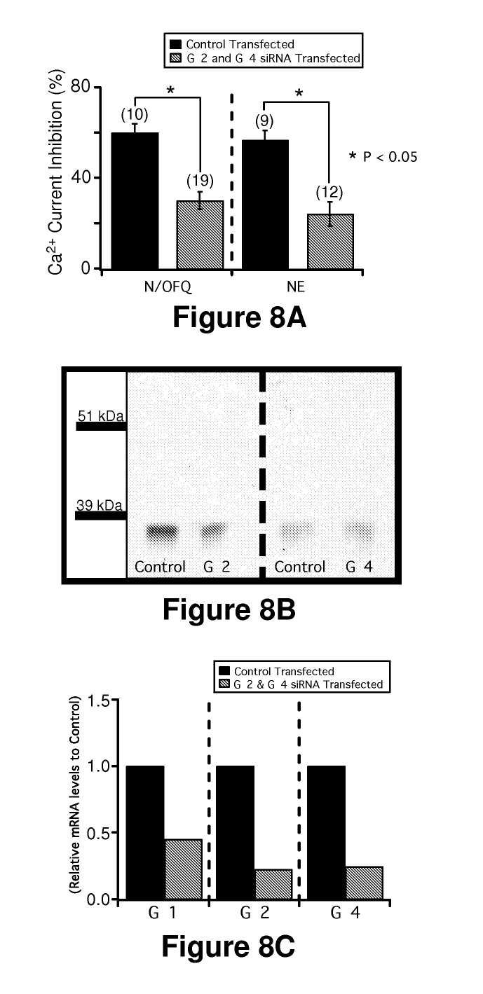

The neurons are dispersed the day prior to electrophysiological recordings by incubating the SG tissue for 60 min in Earle's Balanced Salt Solution containing 0.6 milligrams/milliliter collagenase, 0.4 milligrams/milliliter trypsin and 0.1 milligrams/milliliter DNase at 37° C. The cells are stored in MEM (10% FBS, 1% glutamine and 1% Pen-Strep) prior to recording. The external recording solution contains (in millimolar): TEA-OH 145 millimolar, HEPES 10 millimolar, CaCl₂ 10 millimolar, glucose 15 millimolar and 0.0003 millimolar tetrodotoxin. The pH is 7.4 and the Osmolality is 316-320 milliOsmoles/kilogram. The internal solution contains: N-methyl-D-glucamine (NMG) 80 millimolar, TEA 20 millimolar, EGTA 11 millimolar, CaCl₂ 1 millimolar, HEPES 10 millimolar, Mg-ATP 4 millimolar, Na₂GTP 0.3 millimolar, CsCl 20 millimolar, CsOH 40 millimolar and TRIS-creatine phosphate 14 millimolar. The pH is 7.2 and the osmolality is 293-307 milliOsmoles/kilogram. FIG. 7A is a graph showing the effects of transfection of SG neurons in vitro with siRNA oligonucleotides directed against the G protein Gβ2 by a combination of electroporation+BDM and lipofection+BDM, compared to a control. FIG. 8A is an graph indicating that electrophysiological analysis shows that Gβ2 and Gβ4 functional parameters decrease 3-4 days post siRNA transfection using a combination of electroporation+BDM and lipofection+BDM.

Drugs: Stock solutions of N/OFQ (nociceptin, from Tocris Cookson) and norepinephrine (NE, from Sigma Chem. Co.) are prepared in water and diluted in the external solution to their final concentration prior to use.

The recording protocol consists of a test pulse to +10 mV (prepulse) followed by a large depolarizing conditioning test pulse to +80 mV, a brief return to −80 mV, and followed by a test pulse to +10 mV (postpulse). The peak Ca²⁺ current amplitude is measured isochronally 10 milliseconds after the initiation of the prepulse and postpulse.

Western Blotting

Protein from SG tissue is prepared using a protein lysis buffer (Macherey Nagel). After determination of protein, 20 micrograms of protein are loaded on each lane of NuPAGE® 10% Bis-Tris pre-cast gels (Invitrogen). The gels are run at 200 Volts for 55 minutes. Thereafter, the gels are transferred to PVDF membranes. Detection of Gβ protein subunits is performed with rabbit polyclonal antibodies for Gβ1-β4 (Santa Cruz Biotech.) and goat polyclonal antibody to actin. FIG. 7B is an image showing the effects of transfection of SG neurons in vitro with siRNA oligonucleotides directed against the G protein Gβ2 by a combination of electroporation+BDM and lipofection+BDM, compared to a control. FIG. 8B is an image indicating that Western blot analysis shows that Gβ2 and Gβ4 protein levels decrease 3-4 days post siRNA transfection using a combination of electroporation+BDM and lipofection+BDM.

Quantitative RT-PCR

SG tissue mRNA is obtained with a lysis buffer (Macherey Nagel). TaqMan® Gene Expression Assays (Applied Biosystems) are employed for measuring rat Gβ1, β2, β4 and GAPDH mRNA levels. The assays are run on a 7900HT PCR System (Applied Biosystems) and analyzed on a PC. FIG. 7C is a graph showing the effects of transfection of SG neurons in vitro with siRNA oligonucleotides directed against the G protein Gβ2 by a combination of electroporation+BDM and lipofection+BDM, compared to a control. FIG. 8C is a graph indicating that quantitative RT-PCR analysis shows that Gβ1, Gβ2 and Gβ4 mRNA levels decrease 3-4 days post siRNA transfection using a combination of electroporation+BDM and lipofection+BDM.

### Example 9

A cationic liposome preparation of dimethyldioctadecylammonium bromide (DDAB) and dioleoylphosphatidylethanolamine (DOPE) in a 2:5 w/w ratio can be prepared as described in Lappalainen et al., Pharmaceutical Research, 11(8):1127-1131, 1994. Briefly described, the liposomes can be prepared by mixing DDAB (1.32 milligrams) and DOPE (3.31 milligrams) in chloroform and evaporating to dryness in a rotating evaporator. Three milliliters of sterile water is added and one hour later the mixture is sonicated for 10 minutes.

Electroporation can be carried out with the NEON™ Electroporator (Invitrogen) by preincubating for 15-20 minutes the SG tissue in an RNase- and DNase-free microcentrifuge tube containing the R solution (provided with the NEON Kit), Gβ siRNA (1500 nanomolar) and 2,3-butanedione monoxime (BDM, 2 millimolar). Afterwards, the SG tissue can be drawn up to the 100 microliter electroporator pipet tip and electroporated with a single 20 msec 1000 Volt pulse. The SG tissue can then be placed in a 22 mm dish containing Opti-MEM® supplemented with Gβ siRNA (1500 nM), BDM (2 millimolar) and 10 micromolar final concentration of the DDAB/DOPE liposomes for 4 hours. Following the 4 hour incubation period, the 22 mm dish can be rinsed 3 times with MEM and stored in MEM supplemented with 2% FBS, 1% Pen-Strep, 1% glutamine.

### Example 10

Electroporation can be carried out with the NEON™ Electroporator (invitrogen) by preincubating for 15-20 minutes the SG tissue in an RNase- and DNase-free microcentrifuge tube containing the R solution (provided with the NEON Kit), Gβ siRNA (1500 nanomolar) and 2,3-butanedione monoxime (BDM, 1 millimolar). Afterwards, the SG tissue can be drawn up to the 100 microliter electroporator pipet tip and electroporated with a single 20 millisecond 1000 Volt pulse. The SG tissue can then be placed in a 22 mm dish containing Opti-MEM® supplemented with Gβ siRNA (1500 nM), BDM (1 millimolar) and 10 micromolar final concentration of the DDAB/DOPE liposomes for 4 hours. Following the 4 hour incubation period, the 22 mm dish can be rinsed 3 times with MEM and stored in MEM supplemented with 2% FBS, 1% Pen-Strep, 1% glutamine.

### Example 11

Electroporation can be carried out with the NEON™ Electroporator (Invitrogen) by preincubating for 15-20 minutes the SG tissue in an RNase- and DNase-free microcentrifuge tube containing the R solution (provided with the NEON Kit), Gβ siRNA (1500 nanomolar) and 2,3-butanedione monoxime (BDM, 10 millimolar). Afterwards, the SG tissue can be drawn up to the 100 microliter electroporator pipet tip and electroporated with a single 20 millisecond 1000 Volt pulse. The SG tissue can then be placed in a 22 mm dish containing Opti-MEM® supplemented with Gβ siRNA (1500 nM), BDM (10 millimolar) and 10 micromolar final concentration of the DDAB/DOPE liposomes for 4 hours. Following the 4 hour incubation period, the 22 mm dish can be rinsed 3 times with MEM and stored in MEM supplemented with 2% FBS, 1% Pen-Strep, 1% glutamine.

### Example 12

Electroporation can be carried out with the NEON™ Electroporator (Invitrogen) by preincubating U251 human glioma cells for 15-20 minutes in an RNase- and DNase-free microcentrifuge tube containing the R solution (provided with the NEON Kit), Gβ siRNA (1500 nanomolar) and 2,3-butanedione monoxime (BDM, 10 millimolar). Afterwards, the U251 human glioma cells can be drawn up to the 100 microliter electroporator pipet tip and electroporated with a single 20 millisecond 1000 Volt pulse. The U251 human glioma cells can then be placed in a 22 mm dish containing Opti-MEM® supplemented with Gβ siRNA (1500 nanomolar), BDM (10 millimolar) and 10 micromolar final concentration of the DDAB/DOPE liposomes for 4 hours. Following the 4 hour incubation period, the 22 mm dish can be rinsed 3 times with MEM and stored in supplemented DMEM at 37° C.

### Example 13

Electroporation can be carried out by preincubating U251 human glioma cells for 15-20 minutes in an RNase- and DNase-free microcentrifuge tube containing serum-free DMEM, Gβ siRNA (1500 nanomolar) and 2,3-butanedione monoxime (BDM, 10 millimolar). The cells can be electroporated at 1250 Volts, with a 10 millisecond duration, applied three times (3 pulses). The cells can then be placed in a 22 mm dish containing Opti-MEM® supplemented with Gβ siRNA (1500 nanomolar), BDM (10 millimolar) and 10 micromolar final concentration of the DDAB/DOPE liposomes for 4 hours. Following the 4 hour incubation period, the 22 mm dish can be rinsed 3 times with MEM and stored in supplemented DMEM at 37° C.

### Example 14

Electroporation can be carried out by preincubating SG or SCG neurons for 15-20 minutes in an RNase- and DNase-free microcentrifuge tube containing serum-free DMEM, Gβ siRNA (1500 nanomolar) and 2,3-butanedione monoxime (BDM, 5 millimolar). The cells can be electroporated with a single 20 msec 1000 Volt pulse. The cells can then be placed in a 22 mm dish containing Opti-MEM® supplemented with Gβ siRNA (1500 nM), BDM (5 millimolar) and 10 micromolar final concentration of the DDAB/DOPE liposomes for 4 hours. Following the 4 hour incubation period, the 22 mm dish can be rinsed 3 times with MEM and stored in supplemented DMEM at 37° C.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for material transfer into a cell, comprising:
electroporation of the cell in the presence of the material and 2,3-butanedione monoxime; and
contacting the cell with a complex of a lipid-based carrier and the material, in the presence of 2,3-butanedione monoxime.

2. The method of claim 1, wherein the material is a nucleic acid.

3. The method of claim 1, wherein the cell is in vitro.

4. The method of claim 1, wherein the lipid-based carrier is a liposome.

5. The method of claim 1, wherein the electroporation and contacting the cell with a complex of a lipid-based carrier and the material are performed in any order or simultaneously.

6. The method of claim 1, wherein the nucleic acid is DNA.

7. The method of claim 1, wherein the nucleic acid encodes an inhibitory RNA.

8. The method of claim 1, wherein the nucleic acid is siRNA and/or shRNA.

9. The method of claim 1, wherein the cell is a eukaryotic cell.

10. The method of claim 1, wherein the cell is an excitable cell.

11. A method for nucleic acid transfer into a cell, comprising:
electroporation of the cell in the presence of the nucleic acid and 2,3-butanedione monoxime; and
contacting the cell with a complex of a liposome and the nucleic acid, in the presence of 2,3-butanedione monoxime.

12. The method of claim 11, wherein the cell is in vitro.

13. The method of claim 11, wherein the electroporation and contacting the cell with of a liposome and the nucleic acid are performed in any order or simultaneously.

14. The method of claim 11, wherein the nucleic acid is DNA.

15. The method of claim 11, wherein the nucleic acid encodes an inhibitory RNA.

16. The method of claim 11, wherein the nucleic acid is siRNA and/or shRNA

17. The method of claim 11, wherein the cell is a eukaryotic cell.

18. The method of claim 11, wherein the cell is an excitable cell.

19. A kit for material transfer into a cell, comprising:
an electroporation reagent;
2,3-butanedione monoxime; and
a lipid-based carrier.

20. The kit of claim 19 wherein the electroporation reagent comprises a cell-compatible buffer.
